(19) United States
(12) Patent Application Publication
(10) Pub. No.: US 2012/0143158 A1
(43) Pub. Date: Jun. 7, 2012

(54) ABSORBENT ARTICLE

(75) Inventors: Ho-Hsi Yang, Chia Li Dist. (TW); Yen-Jung Hu, Chia Li Dist. (TW); Chien-Chung Su, Chia Li Dist. (TW); Jung-Chi Tai, Chia Li Dist. (TW)

(73) Assignee: KANG NA HSIUNG ENTERPRISE CO., LTD., Chia Li Dist. (TW)

(21) Appl. No.: 13/113,758

(22) Filed: May 23, 2011

(30) Foreign Application Priority Data
Dec. 6, 2010 (TW) 099142346

(51) Int. Cl. A61L 15/46; A61F 13/51

(52) U.S. Cl. 604/360; 604/378

(57) ABSTRACT

An absorbent article for absorbing a body fluid from a user includes a liquid-impermeable base layer, a liquid-permeable top layer, an absorbent layer disposed between the liquid-impermeable base layer and the liquid-permeable top layer, and a body fluid-collecting layer disposed between the liquid-permeable top layer and the absorbent layer and having a collecting region. The collecting region is formed with a plurality of indentations that are indented in a direction from the liquid-permeable top layer to the liquid-impermeable base layer and that are distributed densely in the collecting region.

### CROSS-REFERENCE TO RELATED APPLICATION

This application claims priority of Taiwanese application no. 099142346, filed on Dec. 6, 2010.

### BACKGROUND OF THE INVENTION

1. Field of the Invention

This invention relates to an absorbent article for absorbing a body fluid from a user.

2. Description of the Related Art

An absorbent article, such as a diaper, a pants-like diaper, a sanitary napkin, etc., generally includes a liquid-impermeable base layer, a liquid-permeable top layer, and an absorbent layer disposed between the liquid-impermeable base layer and the liquid-permeable top layer.

Generally, the absorbent layer has a flat surface. When a relatively large volume of body fluid is excreted, the absorbent layer beneath a body portion where the body fluid is excreted has a limited ability to absorb the large body fluid within a short time, thereby resulting in accumulation of the body fluid on the liquid-permeable top layer, which makes the user uncomfortable and which provides a warm and wet environment for growth of microbes.

Chinese patent application no. 200910208511.1 discloses an absorbent article that includes a base layer and an absorbent layer having a large opening that extends through the absorbent layer and that is used for receiving the body excrement, especially feces, discharged from a user. However, in use, because of an external pressure from the user, a region of the absorbent layer near the large opening is likely to deform and is pressed against the base layer. Therefore, user skin may directly contact the excrement retained on the liquid-impermeable base layer, thereby resulting in irritation for the user's skin. Moreover, the body excrement in the opening is likely to leak out.

### SUMMARY OF THE INVENTION

Therefore, the object of the present invention is to provide an absorbent article that can overcome the aforesaid drawbacks of the prior art.

The absorbent article for absorbing a body fluid from a user according to the present invention includes a liquid-impermeable base layer, a liquid-permeable top layer, an absorbent layer disposed between the liquid-impermeable base layer and the liquid-permeable top layer, and a body fluid-collecting layer disposed between the liquid-permeable top layer and the absorbent layer and having a collecting region. The collecting region is formed with a plurality of indentations that are indented in a direction from the liquid-permeable top layer to the liquid-impermeable base layer and that are distributed densely in the collecting region.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a top schematic view of the first preferred embodiment of an absorbent article of this invention;

FIG. 2 is a fragmentary cross-sectional schematic view of the first preferred embodiment shown in FIG. 1, illustrating a structure and an arrangement of indentations;

FIG. 3 is a fragmentary schematic view of the second preferred embodiment of this invention, illustrating shape and arrangement of the indentations;

FIG. 4 is a fragmentary schematic view of the third preferred embodiment of this invention, illustrating shape and arrangement of the indentations; and

FIG. 5 is a fragmentary schematic view of the fourth preferred embodiment of this invention, illustrating shape and arrangement of the indentations.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

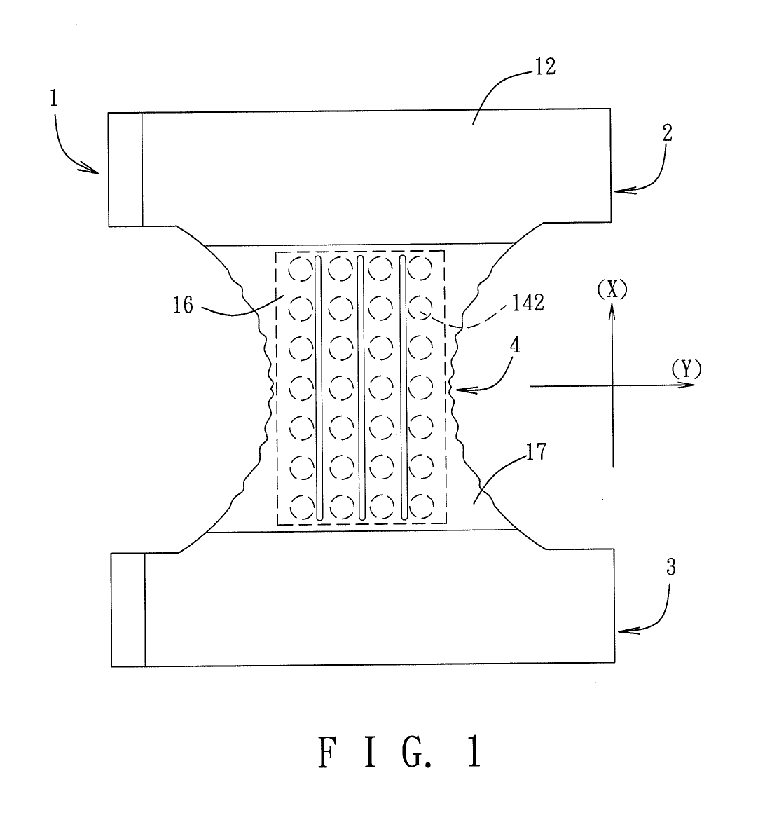

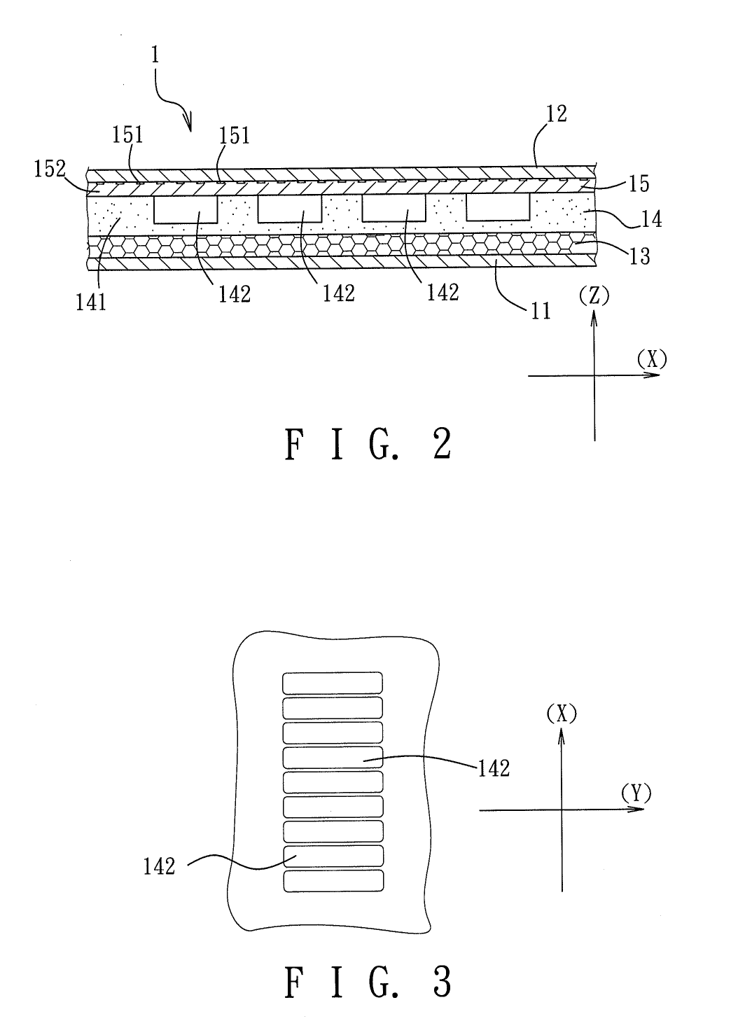

FIGS. 1 and 2 show the first preferred embodiment of an absorbent article 1 according to the present invention. In this embodiment, the absorbent article 1 is exemplified to be in the form of a disposable diaper. However, the absorbent article of this invention can be a sanitary napkin in other embodiments. The absorbent article 1 of the present invention includes a liquid-impermeable base layer 11, a liquid-permeable top layer 12, an absorbent layer 13 disposed between the liquid-impermeable base layer 11 and the liquid-permeable top layer 12, and a body fluid-collecting layer 14 disposed between the liquid-permeable top layer 12 and the absorbent layer 13 and having a collecting region 16 and a non-indented region 17. The non-indented region 17 surrounds the collecting region 16. The stiffness of the collecting region 16 is higher than that of the non-indented region 17.

In this preferred embodiment, the liquid-impermeable base layer 11 is composed of a plastic film which is liquid impermeable but vapor permeable. The liquid-permeable top layer 12 is composed of nonwoven fabric which can provide a better contact feeling for a user. The absorbent layer 13 is composed of absorbent nonwoven fabric that is able to quickly receive liquid from the body fluid-collecting layer 14.

The collecting region 16 has an absorbent body 141 and is formed with a plurality of indentations 142 that are encompassed by the absorbent body 141. The indentations 142 are indented in a direction (Z) from the liquid-permeable top layer 12 to the liquid-impermeable base layer 11, and are distributed densely in the collecting region 16. Preferably, the indentations 142 are evenly distributed in the collecting region 16. In use, the collecting region 16 is disposed beneath the body portion (not shown) where a body fluid discharged, e.g., urethra orifice, anus or ostium vagina. When the body fluid is excreted, the body fluid flows into the indentations 142 through the liquid-permeable top layer 12, and is temporarily received in the indentations 142. The body fluid received in the indentations 142 is then gradually absorbed by the absorbent body 141, the non-indented region 17 and the absorbent layer 13. With the arrangement of the indentations 142, the body fluid, even the fluid with a relatively large volume, can be firstly retained in the indentations 142 so as to prevent leakage of the body fluid. Moreover, because the indentations 142 do not extend through the body fluid-collecting layer 14, i.e., are supported by the absorbent body 141, the stiffness in the collecting region 16 becomes relatively high. Accordingly, in use, the structure of the indentations 142 can be maintained, and thus, the body fluid retained in the indentations 142 will not contact the user. In this preferred embodiment, the body fluid-collecting layer 14 is composed of absorbent nonwoven fabric and the indentations 142 thereof are produced by a pattern drum.

Preferably, the absorbent article 1 further includes a germproof layer 15 that is disposed between the liquid-permeable top layer 12 and the absorbent layer 13. The germproof layer 15 includes a matrix 152 and silver metal particles 151 formed on a surface of the matrix 152 by sputtering. The matrix 152 can be composed of a nonwoven fabric. The silver metal particles 151 formed on the surface of the matrix 152 by sputtering has a better adherence than that formed by coating or dipping, and has greater contact area than that formed by mixing silver powder in the matrix 152, so that the absorbent article 1 has a superior germproof effect. In this embodiment, the germproof layer 15 is disposed between the liquid-permeable top layer 12 and the body fluid-collecting layer 14. Alternatively, the germproof layer 15 can be disposed between the absorbent layer 13 and the body fluid-collecting layer 14.

The absorbent article 1 further comprises front and rear waist portions 2, 3, and a crotch portion 4 disposed between the front and rear waist portions 2, 3 (see FIG. 1). The indentations 142 are formed in the crotch portion 4 and each of the indentations 142 has a first dimension which extends in a direction (X) between the front and rear waist portions 2, 3 and which ranges from 3 cm to 30 cm, a second dimension which extends in a direction (Y) transverse to the direction (X) and which ranges from 1 cm to 8 cm, and a depth which extends in the direction (Z) and which ranges from 0.5 cm to 3 cm.

In this preferred embodiment, each of the indentations 142 has a circular shape, and the indentations 142 are arranged in a matrix.

FIG. 3 shows the shape and the arrangement of the indentations 142 of the second preferred embodiment of the absorbent article of this invention. The absorbent article of the second preferred embodiment has a structure similar to that of the first preferred embodiment, except that each of the indentations 142 has a rectangular shape, and the indentations 142 are arranged in rows along the direction (X).

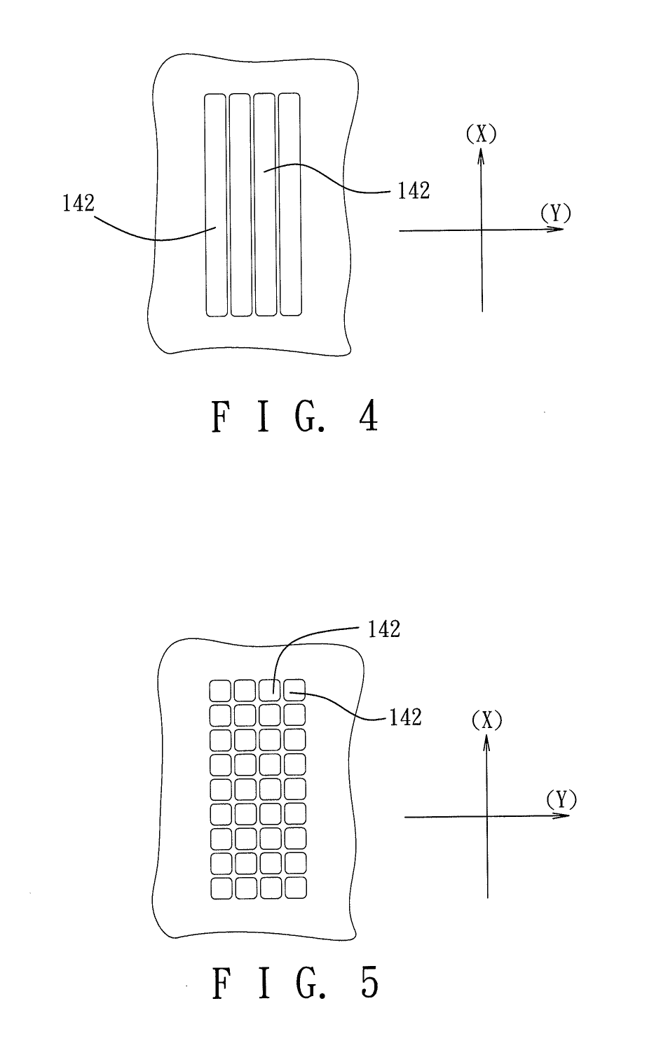

FIG. 4 shows the shape and the arrangement of the indentations 142 of the third preferred embodiment of the absorbent article of this invention. The absorbent article of the third preferred embodiment has a structure similar to that of the first preferred embodiment, except that each of the indentations 142 has a rectangular shape, and the indentations 142 are arranged in rows along the direction (Y).

FIG. 5 shows the shape and the arrangement of the indentations 142 of the fourth preferred embodiment of the absorbent article of this invention. The absorbent article of the fourth preferred embodiment has a structure similar to that of the first preferred embodiment, except that each of the indentations 142 has a square shape, and the indentations 142 are arranged in a matrix.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An absorbent article for absorbing a body fluid from a user, comprising:
a liquid-impermeable base layer;
a liquid-permeable top layer;
an absorbent layer disposed between said liquid-impermeable base layer and said liquid-permeable top layer; and
a body fluid-collecting layer disposed between said liquid-permeable top layer and said absorbent layer and having a collecting region, said collecting region being formed with a plurality of indentations that are indented in a direction (Z) from said liquid-permeable top layer to said liquid-impermeable base layer and that are distributed densely in said collecting region.

2. The absorbent article of claim 1, wherein said body fluid-collecting layer further includes a non-indented region surrounding said collecting region, said collecting region having a higher stiffness than that of said non-indented region.

3. The absorbent article of claim 1, further comprising a germproof layer that is disposed between said liquid-permeable top layer and said absorbent layer and that includes silver metal.

4. The absorbent article of claim 3, wherein said germproof layer is disposed between said absorbent layer and said body fluid-collecting layer.

5. The absorbent article of claim 3, wherein said germproof layer is disposed between said liquid-permeable top layer and said body fluid-collecting layer.

6. The absorbent article of claim 1, wherein said indentations are evenly distributed in said collecting region.

7. The absorbent article of claim 1, further comprising front and rear waist portions and a crotch portion disposed between said front and rear waist portions, said indentations being formed in said crotch portion and having a first dimension which extends in a direction (X) between said front and rear waist portions and ranges from 3 cm to 30 cm, a second dimension which extends in a direction (Y) transverse to said direction (X) and ranges from 1 cm to 8 cm, and a depth which extends in said direction (Z) and ranges from 0.5 cm to 3 cm.

8. The absorbent article of claim 7, wherein each of said indentations has a circular shape, said indentations being arranged in a matrix.

9. The absorbent article of claim 7, wherein each of said indentations has a rectangular shape, said indentations being arranged in rows along one of said directions (X) and (Y).

10. The absorbent article of claim 7, wherein each of said indentations has a square shape, said indentations being arranged in a matrix.
